(19) United States
(12) Patent Application Publication
(10) Pub. No.: US 2012/0123231 A1
(43) Pub. Date: May 17, 2012

(54) MONITORING CARDIAC OUTPUT AND VESSEL FLUID VOLUME

(76) Inventor: Michael O'Reilly, Dana Point, CA (US)

(21) Appl. No.: 13/280,046

(22) Filed: Oct. 24, 2011

Related U.S. Application Data
(60) Provisional application No. 61/412,742, filed on Nov. 11, 2010.

(51) Int. Cl. A61B 5/02; A61B 5/1455

(52) U.S. Cl. 600/340; 600/323; 600/322

(57) ABSTRACT

The present disclosure describes embodiments of a patient monitoring system and methods that include the measure and display of hemoglobin statistics, cardiac output statistic and vessel volume statistics. In an embodiment, total hemoglobin trending, cardiac output, or vessel volume is displayed over a period of time. Statistics can include frequency domain analysis, differences between measurement sites, or further calculations based on concentrations and volume of fluids added to a patient which may be unique for each patient monitored. The total trending and/or statistics can further be used to help control the treatment of a patient, such as being used to control IV administration.

### PRIORITY CLAIM

This application claims the benefit of priority under 35 U.S.C. §119(e) of the following U.S. Provisional Patent Application No. 61/412,742, titled “Monitoring Cardiac Output and Vessel Fluid Volume,” filed on Nov. 11, 2010, and incorporates that application by reference herein in its entirety.

### FIELD OF THE INVENTION

The present disclosure relates to the determination of cardiac output, vessel fluid volume and other cardiovascular related measurements.

### BACKGROUND

During patient care, it is important for a caregiver to know the composition of the patient's blood. Knowing the composition of the patient's blood can provide an indication of the patient's condition, assist in patient diagnosis, and assist in determining a course of treatment. One blood component in particular, hemoglobin, is very important. Hemoglobin is responsible for the transport of oxygen from the lungs to the rest of the body. If there is insufficient total hemoglobin or if the hemoglobin is unable to bind with or carry enough oxygen, then the patient can suffocate. In addition to oxygen, other molecules can bind to hemoglobin. For example, hemoglobin can bind with carbon monoxide to form carboxyhemoglobin. When other molecules bind to hemoglobin, the hemoglobin is unable to carry oxygen molecules, and thus the patient is deprived of oxygen. Also, hemoglobin can change its molecular form and become unable to carry oxygen, this type of hemoglobin is called methemoglobin.

Pulse oximetry systems for measuring constituents of circulating blood have gained rapid acceptance in a wide variety of medical applications including surgical wards, intensive care and neonatal units, general wards, home care, physical training, and virtually all types of monitoring scenarios. A pulse oximetry system generally includes an optical sensor applied to a patient, a monitor for processing sensor signals and displaying results and a patient cable electrically interconnecting the sensor and the monitor. A pulse oximetry sensor has light emitting diodes (LEDs), typically at least one emitting a red wavelength and one emitting an infrared (IR) wavelength, and a photodiode detector. The emitters and detector are attached to a patient tissue site, such as a finger. The patient cable transmits drive signals to these emitters from the monitor, and the emitters respond to the drive signals to transmit light into the tissue site. The detector generates a signal responsive to the emitted light after attenuation by pulsatile blood flow within the tissue site. The patient cable transmits the detector signal to the monitor, which processes the signal to provide a numerical readout of physiological parameters such as oxygen saturation (SpO2) and pulse rate.

Standard pulse oximeters, however, are unable to provide an indication of how much hemoglobin is in a patient's blood or whether other molecules were binding to hemoglobin and preventing the hemoglobin from binding with oxygen. Care givers had no alternative but to measure most hemoglobin parameters, such as total hemoglobin, methemoglobin and carboxyhemoglobin by drawing blood and analyzing it in a lab. Given the nature of non-continuous blood analysis in a lab, it was widely believed that total hemoglobin did not change rapidly.

Advanced physiological monitoring systems utilize multiple wavelength sensors and multiple parameter monitors to provide enhanced measurement capabilities including, for example, the measurement of carboxyhemoglobin (HbCO), methemoglobin (HbMet) and total hemoglobin (Hbt or tHb). Physiological monitors and corresponding multiple wavelength optical sensors are described in at least U.S. patent application Ser. No. 11/367,013, filed Mar. 1, 2006 and titled Multiple Wavelength Sensor Emitters and U.S. patent application Ser. No. 11/366,208, filed Mar. 1, 2006 and titled Noninvasive Multi-Parameter Patient Monitor, both assigned to Masimo Laboratories, Irvine, Calif. (“Masimo Labs”) and both incorporated by reference herein. Pulse oximeters capable of reading through motion induced noise are disclosed in at least U.S. Pat. Nos. 6,770,028, 6,658,276, 6,650,917, 6,157,850, 6,002,952, 5,769,785, and 5,758,644; low noise pulse oximetry sensors are disclosed in at least U.S. Pat. Nos. 6,088,607 and 5,782,757; all of which are assigned to Masimo Corporation, Irvine, Calif. (“Masimo”) and are incorporated by reference herein.

Further, physiological monitoring systems that include low noise optical sensors and pulse oximetry monitors, such as any of LNOP® adhesive or reusable sensors, SofTouch™ sensors, Hi-Fi Trauma™ or Blue™ sensors; and any of Radical®, SatShare™, Rad-9™, Rad-5™, Rad-5v™ or PPO+™ Masimo SET® pulse oximeters, are all available from Masimo. Physiological monitoring systems including multiple wavelength sensors and corresponding noninvasive blood parameter monitors, such as Rainbow™ adhesive and reusable sensors and Rad57™, Rad87™ and Radical-7™ monitors for measuring SpO2, pulse rate, perfusion index, signal quality, HbCO and HbMet among other parameters are also available from Masimo.

In addition to hemoglobin and oxygenation of the blood cells, cardiac output is a critical physiological parameter that may be monitored by a caregiver to ensure adequate performance of the heart and distribution of oxygenated blood throughout a patient's body. A current system for measuring cardiac output called thermodilution involves an invasive technique that requires injecting a bolus of cooled liquid near the heart with a catheter inserted inside the body. In these systems, the catheter is navigated into the arteries and positioned near the heart. Once the catheter is correctly positioned, a bolus of cooled liquid is injected into the artery. The catheter then records the temperature change over time a small distance downstream from the injection site using the same catheter. As the rate of change of temperature in the arteries is proportional to the flow of blood through the arteries, this data may then be used to calculate cardiac output of a patient. This method of determining cardiac output is time consuming and potentially harmful to a patient. Furthermore, it does not allow continuous monitoring and therefore is not useful in providing an alarm or warning to a physician when cardiac output may suddenly begin to drop.

Caregivers utilize information gained from monitoring cardiac output in many different scenarios. For example, surgeons monitor cardiac output during surgery of a patient and if cardiac output suddenly falls, surgeons will add fluid until cardiac output improves. This way, every stroke of the heart will have more fluid to pump, thereby improving cardiac output. This assumes the patient's cardiac output has decreased due to a loss of blood, dehydration or some other reason.

Sometimes, however, a surgeon or other caregiver may add too much fluid to patient in response to falling cardiac output. Excess vessel fluid will put extraordinary pressure on the heart and stretch the muscle out further than is normal. Unfortunately, an overextended heart muscle will not pump as efficiently because the actin and myosin will contract from a less than optimal starting position. This causes cardiac output to decrease, even though there is excess fluid volume in the vessel system. Therefore, over hydration of patients has caused decreased cardiac output in patients which has led to many problems including further distressing of cardiac function and has even lead to death.

### SUMMARY

The present disclosure provides for the measurement, display and analysis of cardiac output in living patients. In an embodiment, this is determined by calculating a rate difference between the increase in Sp0₂ readings taken at a patient's ear or other location near a patient's head from the readings taken at a patient's finger or other place removed from the patient's head after a decrease in the oxygenation of a patient's blood. This method will have the advantage, among others, of being a non-invasive method of determining cardiac output that may therefore, be monitored at more regular intervals.

Additionally, the present disclosure provides for the measurement and analysis of vessel fluid volume in patients. Vessel fluid volume may be determined by monitoring the hemoglobin concentration in a patient's arteries over time after a bolus of fluid has been injected into the body. Therefore, the measurement, display and analysis of total hemoglobin (tHb or Hbt) content in living patients is disclosed herein. In an embodiment, the trend of the total hemoglobin in the arteries after injection of a bolus of fluid is analyzed through, for example, a frequency domain analysis to monitor the increase or decrease in the patient's hemoglobin concentration. In an embodiment, a frequency domain analysis is used to determine a specific signature of the hemoglobin increase. In another embodiment, the total amount of hemoglobin change or increase is determined by the monitor in order to determine the initial and/or final volume in the blood vessels.

The injection of the bolus of fluid will increase the volume of fluid in the blood and therefore decrease the concentration of the hemoglobin. The amount the hemoglobin concentration decreases, however, will depend on the initial volume of fluid in the arteries. The greater the initial volume of fluid in the vessels before the bolus of fluid is introduced, the smaller the change or decrease in concentration of the hemoglobin will result and vise versa. Therefore, while a surgeon is adding fluid in order to hydrate a patient, the surgeon can meanwhile monitor the changes in the hemoglobin concentration to determine the changes in the level of fluid volume in the patient. This will be useful because the surgeon can then determine when enough fluid volume has been added so that the patient has achieved a normal or desired level of hydration and vessel fluid volume.

Monitoring of vessel fluid volume will allow a surgeon to make a more accurate determination about whether addition of fluid to a patient will improve a faltering cardiac output. As mentioned above, cardiac output may be improved by adding fluid if a patient is dehydrated, or has low vessel fluid volume. However, at some point adding more fluid will decrease cardiac output because the heart muscle will be stretched to the point where its pumping is no longer efficient and the cardiac muscle cannot properly and completely contract. Therefore a determination of the vessel fluid volume before adding fluid to remedy a patient undergoing a decrease in cardiac output is desirable.

For example, if a measurement of vessel fluid volume determines that a patient already has an optimal amount of fluid in their vessels, the surgeon will be aware that additional fluid will only serve to decrease cardiac output and will therefore refrain from adding further fluid. Conversely, if a vessel fluid volume measurement determines that the fluid is low in a patient, the surgeon or other caregiver will be aware that additional fluid may increase a patient's cardiac output.

The present disclosure provides for the measurement, display and analysis of hemoglobin content in living patients. It has been discovered that, contrary to the widely held understanding that total hemoglobin does not change rapidly, total hemoglobin fluctuates over time. In an embodiment, the trend of a patient's continuous total hemoglobin (tHb or Hbt) measurement is displayed on a display. In an embodiment, the trend of the total hemoglobin is analyzed through, for example, a frequency domain analysis to determine patterns in the patient hemoglobin fluctuation. In an embodiment, a frequency domain analysis is used to determine a specific signature of the hemoglobin variability specific to a particular patient.

Additionally, exemplary uses of these hemoglobin readings are illustrated in conjunction with dialysis treatment and blood transfusions.

### BRIEF DESCRIPTION OF THE DRAWINGS

The drawings and following associated descriptions are provided to illustrate embodiments of the present disclosure and do not limit the scope of the claims. Corresponding numerals indicate corresponding parts, and the leading digit of each numbered item indicates the first figure in which an item is found.

FIG. 1 illustrates a perspective view of a patient monitoring system in accordance with an embodiment of the disclosure.

FIG. 2 illustrates a block drawing of a patient monitoring system in accordance with an embodiment of the disclosure.

FIG. 3 illustrates a planar view of a patient monitor displaying a sample graph of total hemoglobin versus time as may be displayed by a patient monitoring system in accordance with an embodiment of the disclosure.

FIG. 4 illustrates a planar view of a patient monitor displaying a graph of a frequency domain analysis.

FIG. 5 illustrates a block diagram of a method of monitoring and analyzing a patient's total hemoglobin levels.

FIG. 6 illustrates a perspective view of a patient monitoring system with the capability of analyzing and displaying cardiac output, including a finger oximeter and an ear oximeter sensor, in accordance with an embodiment of the disclosure.

FIG. 7 illustrates a block diagram of a method of monitoring and analyzing a patient's cardiac output.

FIG. 8 illustrates a perspective view of a patient monitoring system with the capability of analyzing and displaying vessel fluid volume, in accordance with an embodiment of the disclosure.

FIG. 9 illustrates a block diagram of a method of determining vessel fluid volume, in accordance with an embodiment of the disclosure.

### DETAILED DESCRIPTION

Aspects of the disclosure will now be set forth in detail with respect to the figures and various embodiments. One of skill in the art will appreciate, however, that other embodiments and configurations of the devices and methods disclosed herein will still fall within the scope of this disclosure even if not described in the same detail as some other embodiments. Aspects of various embodiments discussed do not limit the scope of the disclosure herein, which is instead defined by the claims following this description.

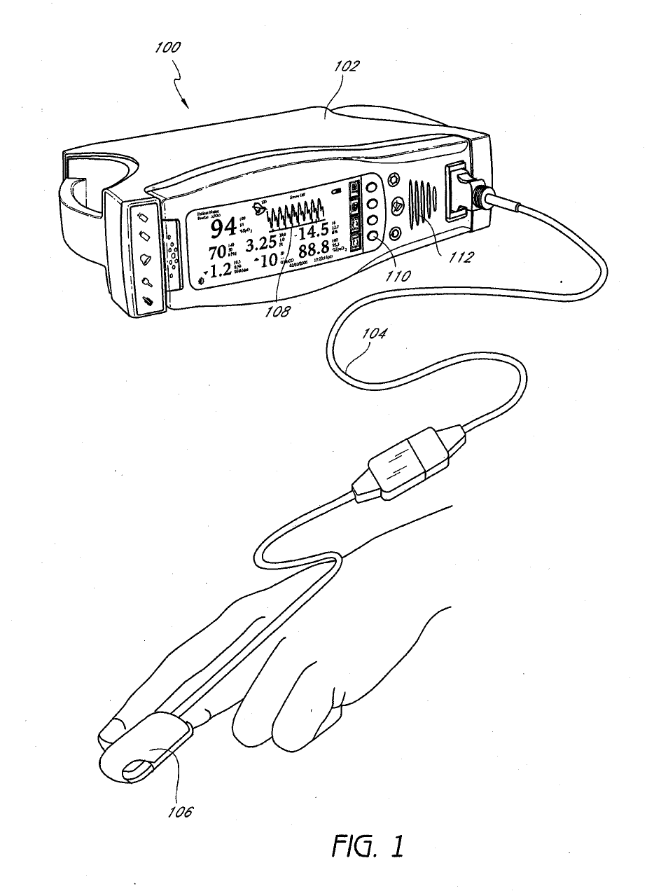

Turning to FIG. 1, an embodiment of a patient monitoring system 100 is illustrated. The patient monitoring system 100 includes a patient monitor 102 attached to at least one sensor 106 by a cable 104. The sensor(s) monitors various physiological data of a patient and sends signals indicative of the parameters to the patient monitor 102 for processing. The patient monitor 102 generally includes a display 108, control buttons 110, and a speaker 112 for audible alerts. The display 108 is capable of displaying readings of various monitored patient parameters, which may include numerical readouts, graphical readouts, and the like. Display 108 may be a liquid crystal display (LCD), a cathode ray tube (CRT), a plasma screen, a Light Emitting Diode (LED) screen, Organic Light Emitting Diode (OLED) screen, or any other suitable display. A patient monitoring system 102 may monitor oxygen saturation (SpO₂), perfusion index (PI), pulse rate (PR), hemoglobin count, cardiac output, vessel fluid volume, and/or other parameters. An embodiment of a patient monitoring system according to the present disclosure is capable of measuring and displaying total hemoglobin trending data and preferably is capable of conducting data analysis as to the total hemoglobin trending. Another embodiment of the patient monitoring system according to the present disclosure is capable of measuring and displaying cardiac output, and displaying a trend in cardiac output. In another embodiment of the present disclosure, the patient monitoring system is capable of measuring and displaying vessel fluid volume including the trend of vessel fluid volume over time.

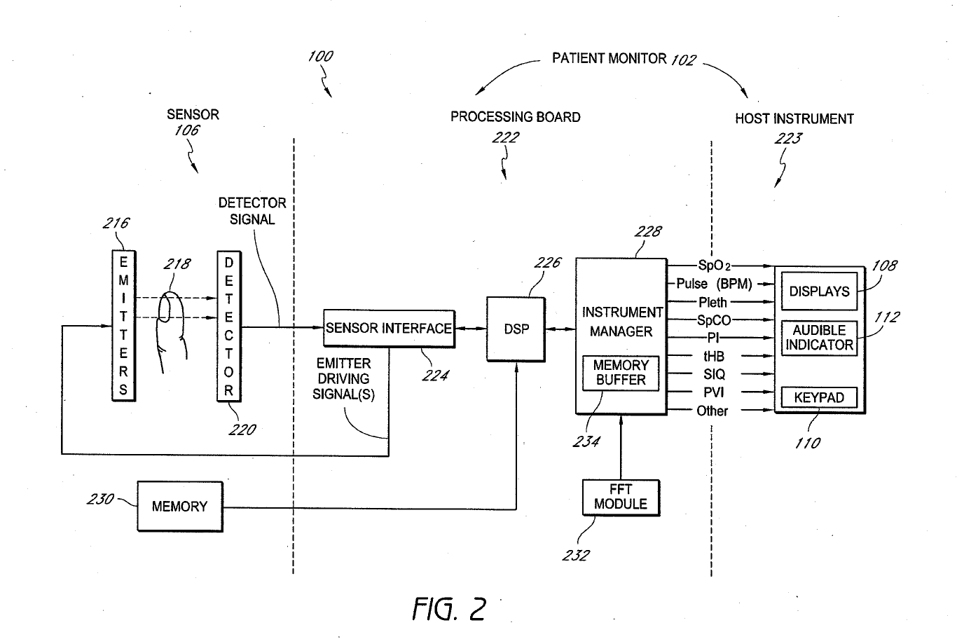

FIG. 2 illustrates details of an embodiment of a patient monitoring system 100 in a schematic form. Typically a sensor 106 includes energy emitters 216 located on one side of a patient monitoring site 218 and one or more detectors 220 located generally opposite. The patient monitoring site 218 is usually a patient's finger (as pictured), toe, ear lobe, or the like. Energy emitters 216, such as LEDs, emit particular wavelengths of energy through the flesh of a patient at the monitoring site 218, which attenuates the energy. The detector(s) 220 then detect the attenuated energy and send representative signals to the patient monitor 102.

Specifically, an embodiment of the patient monitor 102 includes processing board 222 and a host instrument 223. The processing board 222 includes a sensor interface 224, a digital signal processor (DSP) 226, and an instrument manager 228. In an embodiment of the disclosure, the processing board also includes a fast Fourier transform (FFT) module 232. In an embodiment, the FFT module 232 can comprise a special-purpose processing board or chip, a general purpose processor running appropriate software, or the like. The FFT module 232 may further be incorporated within the instrument manager 228 or be maintained as a separate component (as illustrated in FIG. 2).

The host instrument typically includes one or more displays 108, control buttons 110, a speaker 112 for audio messages, and a wireless signal broadcaster. Control buttons 110 may comprise a keypad, a full keyboard, a track wheel, and the like. Additionally embodiments of a patient monitor 102 can include buttons, switches, toggles, check boxes, and the like implemented in software and actuated by a mouse, trackball, touch screen, or other input device.

The sensor interface 224 receives the signals from the sensor 106 detector(s) 220 and passes the signals to the DSP 226 for processing into representations of physiological parameters. These are then passed to the instrument manager 228, which may further process the parameters for display by the host instrument 223. In some embodiments, the DSP 226 also communicates with a memory 230 located on the sensor 106; such memory typically contains information related to the properties of the sensor that may be useful in processing the signals, such as, for example, emitter 216 energy wavelengths. The elements of processing board 222 provide processing of the sensor 106 signals. Tracking medical signals is difficult because the signals may include various anomalies that do not reflect an actual changing patient parameter. Strictly displaying raw signals or even translations of raw signals could lead to inaccurate readings or unwarranted alarm states. The processing board 222 processing generally helps to detect truly changing conditions from limited duration anomalies. The host instrument 223 then is able to display one or more physiological parameters according to instructions from the instrument manager 228, and caregivers can be more confident in the reliability of the readings.

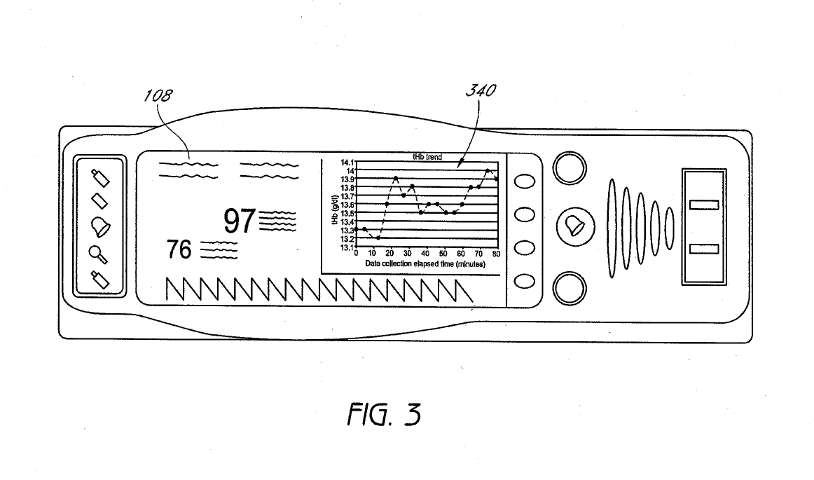

In an embodiment, the patient monitor 102 keeps track of total hemoglobin data over a period of time, such as a few minutes, a few hours, a day or two, or the like. It is important to monitor total hemoglobin over a range of time because it has been discovered that hemoglobin fluctuates over time. In an embodiment, the instrument manager may include a memory buffer 234 to maintain this data for processing throughout a period of time. Memory buffer 234 may include RAM, Flash or other solid state memory, magnetic or optical disk-based memories, combinations of the same or the like. The data for total hemoglobin over a period of time can then be passed to host instrument 223 and displayed on display 108. In an embodiment, such a display may include a graph such as that illustrated by FIG. 3. FIG. 3 illustrates a sample tHb trend graph measuring tHb in g/dL over a period of approximately 80 minutes. In an embodiment, a patient monitor 102 may periodically or continuously update the total hemoglobin display to show the previous hour, previous 90 minutes, or some other desirable time period.

Displaying a current total hemoglobin count, as well as data for a prior time period helps allow a caregiver to determine if the current count is within a normal range experienced by the individual patient. It has also been found that the variations in total hemoglobin count are generally cyclic. It is preferable to display a time period that encompasses at least one complete tHb cycle. As such, a caregiver will be quickly able to see if a total hemoglobin count has fallen above or below the patient's general cyclic range. Additionally, the caregiver may also be able to see if the patient's total hemoglobin count is rising or falling abnormally.

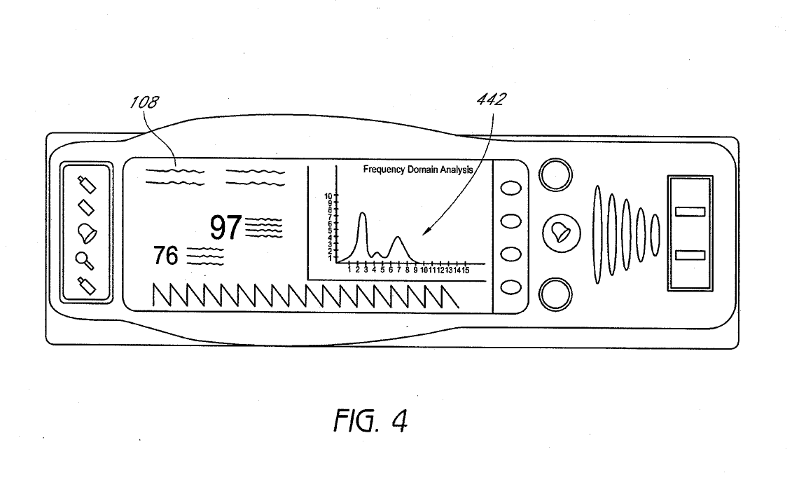

In an embodiment, the trending of the total hemoglobin is additionally or alternatively analyzed through, for example, a frequency domain analysis to determine patterns in the patient hemoglobin fluctuation. Total hemoglobin data from the instrument manager 228 or its memory buffer 234 is passed to the FFT module 232, in an embodiment, to accomplish such an analysis. The FFT module uses one of a number of fast Fourier transform algorithms to obtain the frequencies of various total hemoglobin readings. The resulting data can be graphed and displayed by the host instrument 223's display(s) 108, as shown by example in FIG. 4.

In an embodiment, both total hemoglobin graphs and frequency domain analysis can be displayed on a single patient monitor display 108. In an embodiment, a button 110 or other control allows switching between two such display states. In other embodiments, the display 108 may change automatically, such as periodically or based on a specific event, such as an abnormal change in a patient's total hemoglobin count.

The frequency domain analysis can also be used to identify a specific patient signature, in an embodiment, because hemoglobin frequency variations have been found to be unique or semi-unique between different patients. A portion of the memory buffer 234 may maintain a baseline total hemoglobin frequency data set for comparison to later data readings from the sensor 106. Changes in the frequency analysis may indicate a change in a monitored patient's status. In such an embodiment, a baseline reference graph and a more current frequency domain analysis may be graphed together on a single graph display, on multiple proximate graph displays or display windows, or the like to allow caregivers to recognize changes in the patient's hemoglobin levels over time. For example, in an embodiment, a single graph may include both sets of data graphed in different colors, such as a blue baseline reading and a green more current reading frequency analysis.

The patient monitor 100 may include various alarms that indicate various indications of parameters are falling outside a predetermined range or have reached a level that may endanger the health of the patient. For example, if the cardiac output or fluid volume falls outside a predetermined range an audible or visual or other alert could be triggered on or by the patient monitor 102. In one embodiment, variations between an average value of an indication of a physiological parameter over time and a current reading of an indication of a physiological parameter may, trigger an alert or an alarm if they reach a certain threshold. Such an alert or alarm may be audible and output through audible indicator 112 and/or may alter the display 108. The alarm or alert may incorporate changing colors, flashing portions of a screen, text or audible messages, audible tones, combinations of the same or the like.

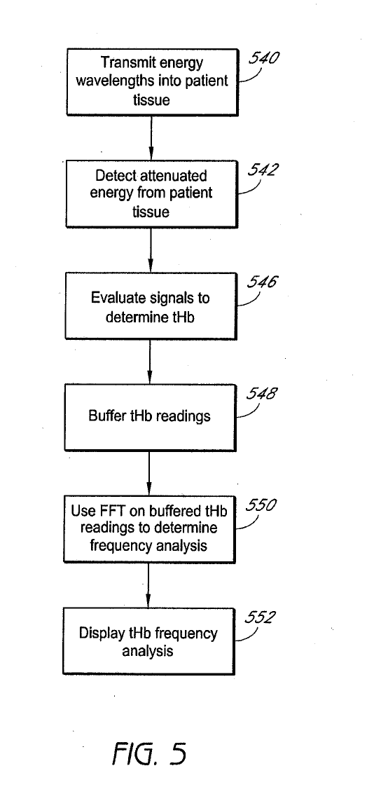

FIG. 5 illustrates an embodiment of a method of obtaining, analyzing, and displaying total hemoglobin data for patient status and analysis as generally described herein. Starting with block 540, energy is transmitted through patient tissue at a measurement site, generally by a sensor 106. The patient tissue attenuates the energy which is then detected at block 542. The detected signals are evaluated to determine a current total hemoglobin count (block 546). This step may include, in an embodiment, filtering noise from the signals, filtering errant readings, and the like. In an embodiment, a buffer stores the total hemoglobin readings for a period of time in (block 548). This allows the patient monitor to display trending data, display the total hemoglobin readings for a period of time, rather than just relatively instantaneous readings, and the like. In an embodiment, the patient monitor analyzes the set of buffered total hemoglobin readings using a Fourier transform, such as a discrete Fourier transform, or more preferably one of many suitable fast Fourier transform algorithms (block 550). This analysis decomposes the sequence of total hemoglobin readings into components of different frequencies. Displaying this frequency analysis (block 552) can help caregivers identify changing conditions for a patient that may indicate worsening or improving health conditions.

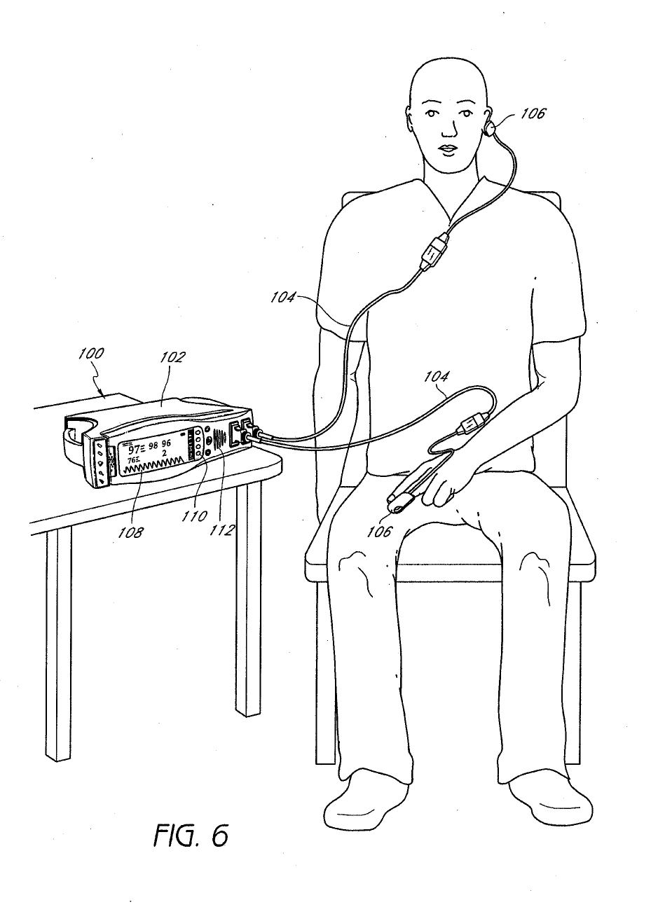

In an embodiment, the patient monitoring system may also determine cardiac output. FIG. 6 illustrates an embodiment of the patient monitoring system utilizing a patient monitor 102 and at least two sensors, including, for example, finger sensor 106 and ear sensor 105, in order to calculate cardiac output. In an embodiment, the patient monitor 102 utilizes the sensors 105, 106 to record the blood oxygenation, or Sp0₂ of a patient in at least two different measurement sites on a patient's body over a period of time. In an embodiment, the patient monitor 102 keeps track of a patient's Sp0₂ data from the two different sites during and after a dip in the oxygenation of a patient's blood. This dip or decrease in blood oxygenation may be induced by asking the patient to hold their breath for a given amount of time.

In another embodiment, a caregiver may use any known method in the art to temporarily reduce the patient's blood oxygenation including manipulating the percentage of oxygen of the gas a patient is inspiring. In an embodiment, a ventilator or other similar device may be used to control the percentage of inspired oxygen or Fi0₂, the patient receives. In an embodiment, while breathing through the device, the Fi0₂ may be lowered to a level that reduces the Sp0₂ of a patient below 100 percent but within a safe range, typically, between 95-99 percent, 88-98 percent, 93-99 percent or other percentages. This can be done by lowering the Fi0₂ until the Sp0₂ reading from a pulse oximeter or other suitable instrument falls within the desired range. At this point, the patient monitor 102 and sensors 105, 106 may begin to record and store the blood oxygenation at two different measurement sites on the patient. Next, the Fi0₂ can be increased while monitoring and storing data related to the differences in aspects of the Sp0₂ levels over time at the two or more measurement sites. This data can then be analyzed to determine the cardiac output of the patient.

The data from the differences in aspects of the Sp0₂ levels over time can be used to determine the cardiac output of a patient. In an embodiment, these differences may amount to the rate of recovery of the blood oxygenation at the at least two different sites. In another embodiment, the difference may the amount of time required to recover a certain percentage of blood oxygenation at the different sites. In another embodiment, the difference may be in a signature or frequency of the recovery of the blood oxygenation at the different sites as measured by the sensors 106.

The patient monitor 102 or other monitoring device can then process and calculate the differences and/or perform further processing and calculations in order to determine the cardiac output of the patient. In an embodiment, the patient monitor 102 could display the cardiac output on the display 108 and provide audible alerts to a caregiver through speaker 112 if the cardiac output dropped below a certain level or moved outside of an acceptable range.

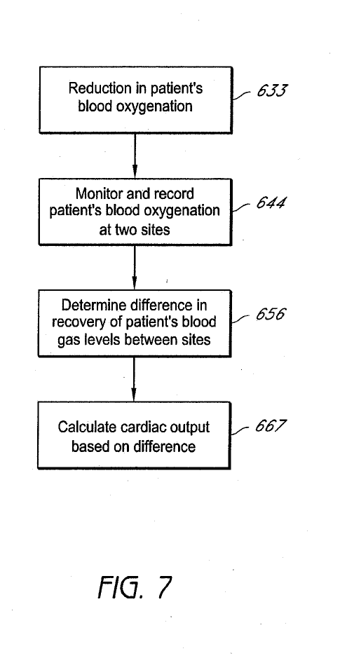

FIG. 7 illustrates an embodiment of a method of determining cardiac output from patient data as generally described herein. Starting with block 633, the patient's blood oxygenation is reduced or lowered by any method known in the art. At that time, the blood oxygenation is monitored by a patient monitor 102 and sensor 106 and recorded or stored in memory in block 644. Next in block 656, the difference between the recovery of the patient's blood gases between different measurement sites (e.g., finger, ear) is determined. The difference may be calculated in many different ways and with a variety of different calculation techniques. These calculations including calculating the difference between the rates of recovery or differences in the amount of time it takes to recover certain percentages of blood oxygenation. Thereafter, the difference in recovery between measurement sites is used to calculate the cardiac output of the patient in block 667. The cardiac output may then be stored in the memory of the patient monitor 102 and/or displayed on display 108.

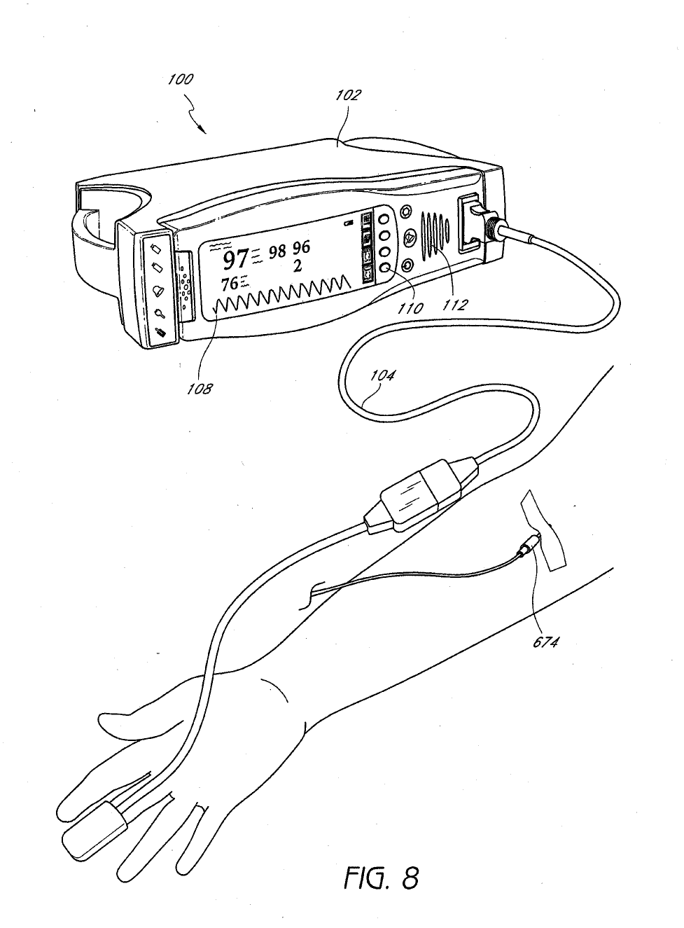

In an embodiment, the patient monitoring system may also determine vessel volume. FIG. 8 illustrates an embodiment of the patient monitoring system utilizing a patient monitor 102, the sensor 106, and a bolus introduction device 674 in order to calculate vessel volume In an embodiment, a caregiver can inject or introduce a bolus of fluid into a patient with the bolus introduction device 674 which can be a syringe, intravenous tube, catheter or any other suitable device known in the art. In an embodiment, the bolus of fluid is introduced into the blood vessel of the patient. In another embodiment, the bolus of fluid is introduced into an artery, vein, or other suitable blood vessel. The fluid may be any suitable fluid known in the art including, saline solution, or other biocompatible solution.

Before and after the injection of the bolus of fluid, the total hemoglobin is recorded with a patient monitoring system over a period of time at a measurement site with sensor 106, as described pursuant to FIG. 5 and generally herein. In an embodiment, the measurement site may be in the general area of a portion of an artery or other blood vessel downstream from the injection site of the bolus of fluid. In an embodiment, the total hemoglobin change after the injection of the bolus of fluid as compared to before the injection is determined. In an embodiment, the patient monitor 102 or other connected processing device may determine the difference in total hemoglobin before and after the injection of the bolus of fluid and at various times after the injection of the bolus of fluid.

The patient monitor 102 or other processor then determines the vessel volume based on the difference in total hemoglobin before and after the introduction of the bolus of fluid. This is determined utilizing principles of chemistry of volume and concentrations of fluid. For example, an unknown volume of a first fluid with a known concentration of a substance dissolved in the first fluid can be determined by the following method. A known volume of a second fluid without the dissolved substance is added to the first fluid. Next, the new concentration of the substance is determined after adding the known volume of second fluid. The volume of the second fluid added can then be multiplied by a ratio of the concentration of the substance before the fluid was added to the concentration of the substance after the second fluid was added. This concept may be applied, partially or fully to calculate the blood vessel volume through total hemoglobin or total hemoglobin concentration as measured by a pulse oximeter and as disclosed herein or other methods known in the art.

However, approximations or references to experimental data may be necessary as the patient body may not imitate a beaker or other container. In one embodiment, a calculation utilized at certain times following the injection may be utilized or at certain points on a curve representing the total hemoglobin over time following the bolus injection. Also, as total hemoglobin will be replaced and red blood cells may be synthesized by the body, if the total hemoglobin is monitored for a certain amount of time to determine the vessel of volume, hemoglobin production by the body may be taken into consideration in calculating the vessel volume.

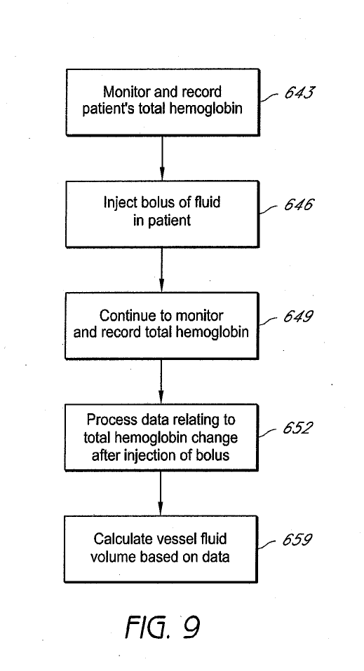

FIG. 9 illustrates an embodiment of a method of determining vessel volume from patient data as generally described herein. First in block 643 the patient monitor 102 and sensor 106 initiates or continues to monitor and record a patient's total hemoglobin or other hemoglobin levels. Next in block 646 a bolus of fluid is introduced to the patient. In one embodiment, the bolus is introduced into the vessel of the patient. In another embodiment, the bolus is introduced into the patient's body in any appropriate tissue. The patient monitoring system then continues to monitor and record the patient's total hemoglobin level on a measurement site on a patient's skin in block 649. In one embodiment, the measurement site may be downstream of the fluid flow of a vessel from the injection site of the fluid bolus. In another embodiment, the measurement site may be in an area removed from the injection site. In another embodiment, the measurement site may be on a vessel upstream from the injection site or any other suitable suit known in the art. Next the data received from the sensor 106 is processed by the patient monitor 102 or other processing device to determine and store the total hemoglobin at all relevant time periods in block 652. In block 659 the vessel fluid volume is calculated based on a formula as disclosed herein or known in the art. In an embodiment, the vessel fluid volume may then be displayed on display 108. If the vessel fluid volume becomes too low, an audible alarm may be issued through speaker 112.

Of course, the foregoing are exemplary only and any IV administered drug, blood, plasma, nutrition, other fluid, or the like that has a tendency to affect hemoglobin levels can be administered and controlled in this manner. One of skill in the art will also understand that the patient monitor and administration devices can be incorporated in a single unit or occur in wired or wirelessly communicating separate units in various embodiments. Administration devices can include not only IV controlling units as discussed, but other devices designed to aid in providing something of need to a patient, such as, for example, a dialysis machine. Similarly, other patient parameters detected by sensor 106 and calculated by patient monitor 102 may also be passed to administration devices or used internally to affect the administration of drugs, blood, nutrition, other fluid, or the like.

Although the foregoing has been described in terms of certain specific embodiments, other embodiments will be apparent to those of ordinary skill in the art from the disclosure herein. Moreover, the described embodiments have been presented by way of example only, and are not intended to limit the scope of the disclosure. Indeed, the novel methods and systems described herein may be embodied in a variety of other forms without departing from the spirit thereof. Accordingly, other combinations, omissions, substitutions, and modifications will be apparent to the skilled artisan in view of the disclosure herein. For example, various functions described as occurring in FFT module 232 may be incorporated within other portions of the processing board 222. Similarly, a patient monitor 102 may not have a distinct processing board 222 and host instrument 223; instead, the various functions described herein may be accomplished by different components within a patient monitor 102 without departing from the spirit of the disclosure. Thus, the present disclosure is not limited by the preferred embodiments, but is defined by reference to the appended claims. The accompanying claims and their equivalents are intended to cover forms or modifications as would fall within the scope and spirit of the disclosure.

What is claimed is:

1. A patient monitoring system comprising:
at least two noninvasive sensors for emitting energy into at least two patient measurement sites and detecting the energy attenuated by the patient measurement sites;
a processing board comprising:
an instrument manager including a memory buffer; and
wherein the instrument manager is adapted to determine an indication of cardiac output using differences in the attenuated energy detected by the at least two sensors at the at least two measurement sites; and
a display adapted to output at least one indication of cardiac output.

2. The patient monitoring system of claim 1 wherein a first of the at least two measurement sites is on or near a patient's head.

3. The patient monitoring system of claim 2 wherein a second of the at least two measurement sites is on a patient's extremity.

4. The patient monitoring system of claim 3 wherein the second of the at least two measurement sites is on a patient's finger.

5. The patient monitoring system of claim 4 wherein the first of the at least two measurements sites is on a patient's ear.

6. The patient monitoring system of claim 1 wherein the difference is a difference in the recovery rate of the blood oxygenation saturation after a desaturation event.

7. The patient monitoring system of claim 6 wherein the difference is a signature in the recovery of the blood oxygenation.

8. The patient monitoring system of claim 1 wherein the difference is a time to reach a certain percentage recovery of the blood oxygenation.

9. The patient monitoring system of claim 1 further comprising:
an administration unit adapted to administer treatment to a patient and in communication with the processing board, wherein the treatment is administered based at least in part on the cardiac output.

10. The patient monitoring system of claim 9 wherein the treatment includes administration of at least one from the following:
a drug;
blood;
plasma;
nutrition; or
an IV fluid.

11. A patient monitor device comprising:
a processing device capable of accepting signals indicative of optical energy attenuated by patient tissue detected from a noninvasive, optical sensor and further capable of interpreting the signals as a measurement of hemoglobin and calculating fluid volume measurements based at least in part on the measurement of hemoglobin;
a memory for storing a plurality of hemoglobin measurements interpreted by the processing device; and
a display for displaying the fluid volume measurements.

12. The patient monitor device of claim 11 wherein the display includes a graph of a plurality of fluid volume versus time.

13. The patient monitor device of claim 11 further comprising a mathematical module adapted to analyze the plurality of hemoglobin measurements to determine the fluid volume for display by the display.

14. The patient monitor device of claim 13 wherein the mathematical module comprises an algorithm based on the concentration of hemoglobin before a bolus of fluid is introduced into a patient's vessels, an amount of the bolus of fluid introduced into a patient's vessels, and the concentration of hemoglobin after the bolus of fluid is introduced into a patient's vessels.

15. The patient monitor device of claim 14 wherein the mathematical module further comprises an approximation based on experimental data.

16. A method for monitoring patient cardiac output levels, the method comprising:
emitting energy into at least two patient measurement sites for attenuation by the at least two measurement sites;
detecting attenuated energy from the at least two measurement sites;
determining a plurality of indications of differences of blood oxygenation between the two measurements sites from the detected attenuated energy over a period of time;
calculating an indication of cardiac output based on the differences of blood oxygenation; and
displaying the indications of cardiac output.

17. The method for monitoring cardiac output levels of claim 16 further comprising the step of storing at least some of the plurality of indications of the differences in blood oxygenation in a buffer.

18. The method for monitoring cardiac output levels of claim 16 further comprising the steps of:
calculating a frequency analysis of the plurality of indications of the differences in blood oxygenation; and
displaying said frequency analysis.

19. The method for monitoring patient cardiac output levels of claim 16 wherein the differences in blood oxygenation represent differences in the recovery rates of blood oxygenation between the measurement sites.

20. A method for treating a patient based on determined vessel volume levels, the method comprising:
emitting energy into a patient measurement site for attenuation by the measurement site;
detecting attenuated energy from the measurement site;
determining a plurality of indications of total hemoglobin from the detected attenuated energy over a period of time;
determining a measure of vessel volume based on the indications of total hemoglobin; and
electronically determining a treatment based at least in part on the measure of vessel volume; and
administering the treatment.

21. The method for treating a patient of claim 20 wherein the step of determining a treatment includes at least one of a rate or amount of an IV treatment.
